(19) United States
(12) Patent Application Publication
(10) Pub. No.: US 2012/0129834 A1
(43) Pub. Date: May 24, 2012

(54) SEROTONIN RECEPTOR ANTAGONISTS FOR USE IN THE TREATMENT OF HUNTINGTON'S DISEASE

(75) Inventors: ROBERT HUGHES, Berkeley, CA (US); LISA ELLERBY, Novato, CA (US)

(73) Assignee: BUCK INSTITUTE FOR RESEARCH ON AGING, Novato, CA (US)

(21) Appl. No.: 13/234,891

(22) Filed: Sep. 16, 2011

Related U.S. Application Data
(60) Provisional application No. 61/386,415, filed on Sep. 24, 2010.
(60) Provisional application No. 61/384,151, filed on Sep. 17, 2010.

(51) Int. Cl. A61K 31/4535; A61K 31/451; A61P 25/00; A61K 31/4545; A61P 25/28; A61K 31/553; A61P 25/14

(52) U.S. Cl. 514/211.13; 514/325; 514/324; 514/318

(57) ABSTRACT

Methods are provided for the treatment and/or prophylaxis of Huntington's disease. In various embodiments the methods involve administration of one or more serotonin receptor antagonists. In certain embodiments the serotonin receptor antagonists include, but are not limited to loxapine, and/or a loxapine analogue, and/or cyproheptadine, and/or a cyproheptadine analogue (e.g., pizotifen).

### CROSS-REFERENCE TO RELATED APPLICATIONS

This application claims benefit of and priority to U.S. Ser. No. 61/386,415, filed Sep. 24, 2010, and to U.S. Ser. No. 61/384,151, filed Sep. 17, 2011, both of which are incorporated herein by reference in their entirety for all purposes.

### STATEMENT OF GOVERNMENTAL SUPPORT

[Not Applicable]

### FIELD OF THE INVENTION

This invention pertains to the field of Huntington's disease and associated cognitive, psychiatric, and motor disorders. In particular this invention pertains to the treatment of Huntington's disease and/or to the treatment of pre- or asymptomatic Huntington's subjects to reduce or prevent cognitive and other dysfunctions associated with later disease progression.

### BACKGROUND

The clinical management of numerous neurological disorders has been frustrated by the progressive nature of degenerative, traumatic, or destructive neurological diseases and the limited efficacy and serious side-effects of available pharmacological agents. Conditions such as Huntington's disease, Alzheimer's disease, severe seizure disorders (e.g., epilepsy and familial dysautonomia), as well as injury or trauma to the nervous system have eluded most conventional pharmacological attempts to alleviate or cure the conditions.

Huntington's disease has proven particularly elusive to conventional pharmacological treatments. Huntington's disease is a progressive degenerative disease of the basal ganglia that is inherited as an autosomal dominant trait. The onset of Huntington's disease, an autosomal dominant, neurodegenerative disorder occurs at an average age of 35 to 40 years but can occur in persons as young as two years old or as old as 80 years.

The onset is insidious and is characterized by abnormalities of coordination, movement, and behavior. Movement abnormalities include restlessness, mild postural abnormalities, and quick jerking movements of the fingers, limbs, and trunk. The movement abnormalities may be accompanied by substantial weight loss. Depression is common, and cognitive abnormalities and inappropriate behavior may develop. In contrast to the choreic movements typical of onset in adults, juvenile patients may exhibit rigidity, tremor, and dystonia. In the course of eight to 15 years, the disorder can progress to complete incapacitation, with most patients dying of aspiration pneumonia or inanition.

Huntington's disease was the first major inherited disorder with an unidentified basic defect to be linked with a DNA marker (Gusella et al. (1983) Nature 306: 234). The product of this gene, designated huntingtin, contains more than 3000 amino acids and is encoded by 10,366 bases at 4p16.3 (Huntington's Disease Collaborative Research Group (1993) Cell 72: 971). Although knowledge of the underlying molecular basis for Huntington's disease has increased in recent years, pharmacological treatments based on this molecular knowledge has been limited.

### SUMMARY

Methods are provided for the treatment and/or prophylaxis of Huntington's disease (HD). In certain embodiments the methods involve the use of one or more serotonin receptor antagonists (e.g., at the 5-HT1 and/or 5-HT2A receptor). In various embodiments the serotonin receptor antagonists include, but are not limited to loxapine, and/or a loxapine analogue, and/or cyproheptadine, and/or a cyproheptadine analogue (e.g., pizotifen).

In certain embodiments therapeutic methods are provided for ameliorating one or more symptoms of Huntington's disease in a mammal (e.g., a non-human mammal, a human). The methods typically involve administering, or causing to be administered, to the mammal one or more agents selected from the group consisting of loxapine, a lopaxine analogue, cyproheptadine, and a cyproheptadine analogue in an amount sufficient to mitigate symptoms of the disease.

In certain embodiments prophylactic methods are provided, e.g., method to reducing the risk, lessen the severity, and/or delay the onset or progression of Huntington's disease in a mammal (e.g., a human). The prophylactic methods typically involve administering, or causing to be administered, to the mammal (e.g., an asymptomatic mammal, a mammal diagnosed as at risk for HD, a mammal in early stage HD, etc.) an effective regime of one or more agents selected from the group consisting of loxapine, a lopaxine analogue, cyproheptadine, and a cyproheptadine analogue thereby reducing the risk, lessening the severity, and/or delaying the progression or onset of the disease.

In certain embodiments the agents used in the prophylactic and/or therapeutic methods comprise cyproheptadine and/or a cyproheptadine analogue. In certain embodiments the cyproheptadine analogue comprises pizotifen. In certain embodiments the cyproheptadine analogue comprises a compound selected from group consisting of the compounds listed in FIG. 2. In certain embodiments the cyproheptadine analogue comprises a compound selected from the group consisting of perithialene, azatadin, ketotifen, pimetixen, and azanator. In certain embodiments the agents the agents used in the prophylactic and/or therapeutic methods comprise loxapine and/or a loxapine analogue. In certain embodiments the loxapine analogue is not clozapine and/or is not a clozapine analogue. In certain embodiments the loxapine analogue is isoloxapine. In certain embodiments the loxapine analogue comprises a compound selected from the group consisting of compounds listed in FIGS. 4-13. In certain embodiments the agent is formulated in a pharmaceutically acceptable excipient. In certain embodiments the agent is formulated as a pharmaceutically acceptable salt, ester, amide, solvate, hydrate, or prodrug thereof. In certain embodiments the agent is formulated for delivery via a modality selected from the group consisting of isophoretic delivery, transdermal delivery, aerosol administration, administration via inhalation, oral administration, intravenous administration, and rectal administration. In various embodiments the agent is administered via a route selected from the group consisting of isophoretic delivery, transdermal delivery, aerosol administration, administration via inhalation, oral administration, intravenous administration, and rectal administration. In various embodiments of the therapeutic methods the mammal is human (e.g., a human diagnosed as having Huntington's disease). In certain embodiments the mammal is a human having the Huntington mutation.

In various embodiments of the prophylactic methods, the mammal is human diagnosed as being at risk for Huntington's disease (e.g., a human that has a familial risk for Huntington's disease, a human diagnosed as having a Huntington mutation, etc.). In various embodiments the mammal does not have, and/or is not under treatment for, and/or is not identified as at risk for one or more conditions selected from the group consisting of an allergy, headache or migraine, depression, Parkinson's disease, schizophrenia, psychosis or other neuropsychiatric disorders, and a sleep disorder. In certain embodiments the mammal does not have and/or is not diagnosed as having HD and is not under treatment for, and/or is not identified as at risk for one or more conditions selected from the group consisting of an allergy, headache or migraine, depression, Parkinson's disease, schizophrenia, psychosis or other neuropsychiatric disorders, and a sleep disorder. In certain embodiments the mammal does not have and/or is not under treatment for, and/or is not at risk for one or more conditions such as headache, and/or vascular headache including migraine and cluster headache. In certain embodiments the mammal does not have and/or is not under treatment for an allergy and/or is not administered an antihistamine. In certain embodiments the mammal does not have a neurological disease or disorder other than Huntington's disease and/or is not diagnosed as having a neurological disease or disorder other than Huntington's disease. In certain embodiments the mammal does not have a Huntington's related sleep disorder.

In various embodiments the ameliorating one or more symptoms of Huntington's disease or reducing the risk, lessen the severity, and/or delay the onset or progression of Huntington's disease comprises inhibiting neural cell death and/or restoring neurological cell function. In various embodiments the ameliorating one or more symptoms of Huntington's disease or reducing the risk, lessen the severity, and/or delay the onset or progression of Huntington's disease comprises inhibiting the onset, progression, or severity of a Huntington's related movement disorder, and/or a Huntington's related cognitive disorder, and/or a Huntington's related psychiatric disorder. In various embodiments the ameliorating one or more symptoms of Huntington's disease or reducing the risk, lessen the severity, and/or delay the onset or progression of Huntington's disease comprises inhibiting the onset, progression, or severity of one or more symptoms selected from the group consisting of clumsiness, jaw clenching (bruxism), loss of coordination and balance, slurred speech, swallowing and/or eating difficulty, uncontrolled continual muscular contractions (dystonia), walking difficulty, stumbling, and falling, loss of memory, loss of concentration, inability to recognize familiar objects, hostility/irritability, inability to take pleasure in life (anhedonia), lack of energy, delusions, hallucinations, inappropriate behavior (e.g., unprovoked aggression), and paranoia. In various embodiments the ameliorating one or more symptoms of Huntington's disease or reducing the risk, lessen the severity, and/or delay the onset or progression of Huntington's disease comprises an improvement in the cognitive abilities of the mammal and/or a perceived improvement in quality of life by the human. In certain embodiments the mammal is a human and progression from an asymptomatic state to a symptomatic state is prevented or delayed. In various embodiments the administering is over a period of at least three weeks and/or over a period of at least 3 months, or at least 6 months, or at least 12 months, or at least 2 years, or at least 5 or 10 years.

Also provided are methods of ameliorating one or more symptoms of Huntington's disease in a mammal, where the methods involve administering to the mammal one or more serotonin receptor antagonists in an amount sufficient to mitigate symptoms of the disease. In various embodiments the agents do not include Risperidone. In certain embodiments the symptoms do not include choreoathetosis of Huntington's disease. In certain embodiments the agents are selected from the group consisting of loxapine, a lopaxine analogue, cyproheptadine, and a cyproheptadine analogue, e.g. as described herein. In certain embodiments the mammal does not have, and/or is not under treatment for, and/or is not identified as at risk for one or more conditions selected from the group consisting of an allergy, headache or migraine, depression, Parkinson's disease, schizophrenia, psychosis or other neuropsychiatric disorders, and a sleep disorder. In certain embodiments the mammal does not have a neurological disease or disorder other than Huntington's disease. In certain embodiments the mammal does not have a Huntington's related sleep disorder. In certain embodiments the mammal is not diagnosed as having or at risk for a neurological disease or disorder other than Huntington's disease.

In various embodiments any of the methods disclosed herein exclude the use of respiridone for the treatment or prophylaxis of one or more symptoms of HD (e.g., choreoathetosis of Huntington's disease). In various embodiments the methods exclude loxapine and any/or of the loxapine analogues described in U.S. Patent Publication 2006/0286167 or incorporated therein by reference.

### Definitions

As used herein, “administering” refers to local and/or systemic administration, e.g., including enteral and parenteral administration. Routes of administration for the active agent(s) described herein include, but are not limited to oral (“p.o.”) administration, administration as a suppository, topical contact, intravenous (“iv”) administration, intraperitoneal (“ip”) administration, intramuscular (“im”) administration, intralesional administration, nasal administration, subcutaneous (“sc”) administration, the implantation of a slow-release and/or regulated release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, ionophoretic and intracranial. Other modes of delivery include, but are not limited to, the use of liposomal formulations, intravenous infusion, transdermal patches, etc.

The terms “systemic administration” and “systemically administered” refer to a method of administering one or more compound(s) or composition(s) to a mammal so that the compound(s) or composition(s) are delivered to sites in the body, including the targeted site of pharmaceutical action, via, for example, the circulatory system. Systemic administration includes, but is not limited to, oral, intranasal, rectal and parenteral (i.e., other than through the alimentary tract, such as intramuscular, intravenous, intra-arterial, transdermal and subcutaneous) administration.

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

The phrase “effective amount” means a dosage or dosage regimen sufficient to produce a desired result.

The term “coadministering” or “concurrent administration”, when used, for example with respect to the active agent(s) described herein (e.g., serotonin receptor antagonists such as loxapine and/or loxapine analogues, and/or cyproheptadine and/or cyproheptadine analogues) and another active agent (e.g. an antipsychotic or mood stabilizer), refers to administration of the serotonin receptor antagonist and the other active agent such that both can simultaneously achieve a physiological effect. The two agents, however, need not be administered together. In certain embodiments, administration of one agent can precede administration of the other. Simultaneous physiological effect need not necessarily require presence of both agents in the circulation at the same time. However, in certain embodiments, coadministering typically results in both agents being simultaneously present in the body (e.g. in the plasma) at a significant fraction (e.g. 20% or greater, preferably 30% or 40% or greater, more preferably 50% or 60% or greater, most preferably 70% or 80% or 90% or greater) of their maximum serum concentration for any given dose.

As used herein, the terms “treating” and “treatment” refer to delaying the onset of, retarding or reversing the progress of, reducing the severity of, or alleviating or preventing either the disease or condition to which the term applies, or one or more symptoms of such disease or condition.

The term “mitigating” in a therapeutic context refers to reduction or elimination of one or more symptoms of that pathology or disease, while mitigation in a prophylactic context refers to a reduction in the rate or delay of onset or ultimate severity of one or more symptoms of that pathology or disease, and/or the prevention of that pathology or disease. Symptoms of Huntington's disease include, but are not limited to one or more of the following: Jerky, random, and uncontrollable movements (chorea) (see, e.g., Walker (2007) Lancet, 369(9557): 218). Chorea may be initially exhibited as general restlessness, small unintentionally initiated or uncompleted motions, lack of coordination, or slowed saccadic eye movements. The clear appearance of symptoms such as rigidity, writhing motions or abnormal posturing appear as the disorder progresses and are signs that the system in the brain that is responsible for movement has been affected. Psychomotor functions become increasingly impaired, such that any action that requires muscle control is affected. Common consequences are physical instability, abnormal facial expression, and difficulties chewing, swallowing and speaking Eating difficulties commonly cause weight loss and may lead to malnutrition. Sleep disturbances are also associated symptoms. In certain instances, particularly in juvenile HD rigidity can be a dominant symptom. Other symptoms include, but are not limited to seizures and the impairment of cognitive abilities (especially executive functions which include planning, cognitive flexibility, abstract thinking, rule acquisition, initiating appropriate actions and inhibiting inappropriate actions). In certain embodiments memory deficits appear in which impairments range from short-term memory deficits to long-term memory difficulties, including deficits in episodic (memory of one's life), procedural (memory of the body of how to perform an activity) and working memory and ultimately cognitive problems can tend to worsen over time, ultimately leading to dementia *(a subcortical dementia syndrome as distinguished from the typical effects of cortical dementias e.g. Alzheimer's disease). Other symptoms include, but are not limited to cellular symptoms such as the formation of Htt protein aggregates and/or the accumulation of such aggregates into inclusion bodies (in the cell nucleus and/or cytoplasm), particularly in neurons. Other symptoms can also include reductions in striatal area/volume in the brain.

As used herein, the phrase “consisting essentially of” refers to the genera or species of active pharmaceutical agents included in a method or composition, as well as any excipients inactive for the intended purpose of the methods or compositions. In some embodiments, the phrase “consisting essentially of” expressly excludes the inclusion of one or more additional active agents other than the active agents described herein (e.g., serotonin receptor antagonists such as loxapine and/or loxapine analogues, and/or cyproheptadine and/or cyproheptadine analogues).

The terms “subject,” “individual,” and “patient” interchangeably refer to a mammal, preferably a human or a non-human primate, but also domesticated mammals (e.g., canine or feline), laboratory mammals (e.g., mouse, rat, rabbit, hamster, guinea pig) and agricultural mammals (e.g., equine, bovine, porcine, ovine). In various embodiments, the subject can be a human (e.g., adult male, adult female, adolescent male, adolescent female, male child, female child) under the care of a physician or other health worker in a hospital, psychiatric care facility, as an outpatient, or other clinical context. In certain embodiments the subject may not be under the care or prescription of a physician or other health worker.

### BRIEF DESCRIPTION OF THE DRAWINGS

FIG. 1 illustrates the structures of cyproheptadine and pizotifen.

FIG. 2 illustrates cyproheptadine and various cyproheptadine analogs.

FIG. 3 illustrates lopaxine.

FIG. 4 illustrates various loxapine analogues (compounds 1-12).

FIG. 5 illustrates various loxapine analogues (compounds 13-21).

FIG. 6 illustrates various loxapine analogues (compounds 22-30).

FIG. 7 illustrates various loxapine analogues (compounds 31-39).

FIG. 8 illustrates various loxapine analogues (compounds 40-48).

FIG. 9 illustrates various loxapine analogues (compounds 49-57).

FIG. 10 illustrates various loxapine analogues (compounds 58-66).

FIG. 11 illustrates various loxapine analogues (compounds 67-74).

FIG. 12 illustrates various loxapine analogues (compounds 75-80).

FIG. 13 illustrates various loxapine analogues (compounds 81-88).

FIG. 14 illustrates inhibition of caspase activation by RNAi against 5-HT receptors HTRF1 and HTR2C in HEK293T cells expressing WT (23Q) and mutant (141Q) Htt. Bars indicate relative caspase 3/7 activity after serum deprivation (normalized to cells expressing Htt141Q treated with a non-targeting siRNA pool). NT (non-targeting) RNAi is the negative control. Caspase-3 RNAi is the positive control. NT vs. NTR1F or HTR2C RNAi treatment is significant (*p<0.05).

FIG. 15 illustrates inhibition of Caspase activation by RNAi against 5-HT receptors HTRF1 and HTR2C in STHdh7Q/7Q and STHdh111Q/111Q knock-in cells. Bars indicate caspase 3/7 activity in cells treated with a non-targeting (NT) siRNA pool. NT (non-targeting) RNAi is the negative control. Caspase-3 RNAi is the positive control. NT vs. NTR1F or HTR2C RNAi treatment is significant (**p<0.01).

FIG. 16 shows data for the top hits from screening the Prestwick library of bioactive compounds for suppressors of cell death in serum deprived STHdh111Q/111Q cells. Viability as measured by ATP levels (normalized to cell number) in cells after 24 h of serum deprivation. Data are show as percent DMSO control (100%). Of the six hits identified as being >110 of control, five are known 5-HTR antagonists. (n=4 in all cases; * indicates p<0.05 as compared to 100%).

FIG. 17 illustrates inhibition of cell death, i.e. ATP levels (upper panel) and caspase activation (lower panel) by 5-HTR antagonists in STHdh111Q/111Q cells (n=3/dose).

FIG. 18 illustrates expression of 5-HTR family members in HD models; STHdh cells and R6/2 mouse mRNA levels as measured by real-time PCR (ABI LIGHTCYCLER®) for 5-HTR genes. Data are normalized to actin mRNA. Upper panel shows expression levels in the striatum of the R6/2 mice expressing 130Q (“HD”) and non-transgenic littermate controls (“WT”). Lower panel shows relative expression in STHdh111Q and STHdh7Q cells in the presence or absence (24 h; SFM)) of serum. n=4 in all cases. For clarity, the y-axes are shown with discontinuous scales.

FIG. 19 shows rotarod performance of R6/2 mice treated with 10 mg/kg Pizotifen. Average latency to fall is shown for control and treatment groups on subsequent days of testing. (n=15 saline, 14 pizotifen treated mice); *p<0.05, **p<0.005, ***p<0.0005.

FIG. 20 shows a dose dependent response of pizotifen on correction of rotarod.

FIG. 21 shows that the drug (e.g., pizotifen 7.5 mg/kg) doesn't improve or worsen weight loss in the mouse model. It demonstrates that the drug is not protective against weight loss.

FIG. 22 shows that striatal area is preserved in pizotifen treated mice. Top panel shows striatal area in sections from animals treated with ntg, 0.9% saline, and Pizotifen (7.5 mg/kg). Middle: Ratio of striatal area/hemisphere area. Bottom: Mean striatal area (mm²).

FIG. 23 shows striatal, but not cortex activation of the ERK pathway in response to pizotifen.

FIG. 24, panels A-C, show that DARP-32 decrease (a measure of disease progression) is rescued by pizotifen.

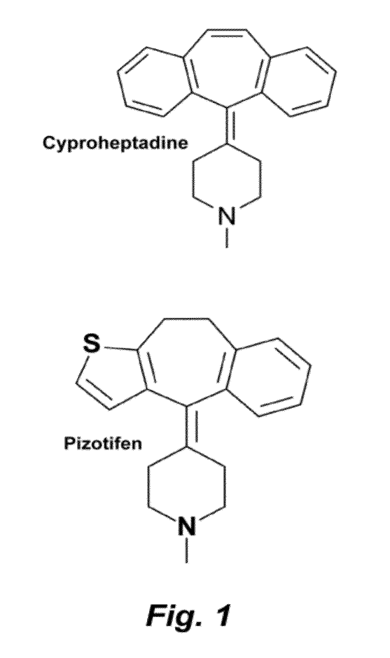

### DETAILED DESCRIPTION

The methods and compositions described herein pertain to the discovery that serotonin antagonists can be used to ameliorate one or more symptoms of Huntington's disease (HD) and can also be available as a prophylactic therapy. In particular embodiments the serotonin receptor antagonists include, but are not limited to loxapine (IUPAC name: 2-Chloro-11-(4-methylpiperazin-1-yl)dibenzo[b,f][1,4]oxazepine) and/or analogues there of and/or cyproheptadine (IUPAC name 4-(5H-dibenzo[a,d]cyclohepten-5-ylidene)-1-methylpiperidine hydrochloride), and/or analogues thereof. One cyproheptadine analogue of particular use is pizotifen (IUPAC name: 4-(1-methyl-4-piperidylidine)-9,10-dihydro-4H-benzo-[4,5]cyclohepta[1,2]-thiophene) and/or its derivatives and analogues.

In order to identify approved drugs that could have disease-modifying activity in Huntington's disease (HD), we used a mammalian cell-based assay of mutant huntingtin-mediated cell death to screen the Prestwick Chemical library. This library is composed of 1,200 marketed drugs with favorable safety, bioavailability and efficacy properties. Our screen identified several compounds that could reproducibly suppress huntingtin-mediated cell death in a dose-dependent manner. Strikingly, three of these hits loxapine, cyproheptadine and pizotifen are approved serotonin receptor antagonist drugs.

In an independent genome-scale RNAi screen for suppressors of cell death using this same cell-based model, we also obtained the targets of these hits as modifiers including HTR2C and HTR1F. These data demonstrate that serotonin receptors and ligands are relevant to HD. On the basis of these observations, we selected Pizotifien for testing in the R6/2 mouse model of HD. Pizotifen was selected because of its safety profile and CNS penetrance. In two independent studies, we observed that this drug was able to robustly correct motor deficits (i.e. Rotarod performance) in R6/2 to near wild-type levels. Open field behavior analysis indicated that the general activity level of treated animals was normal. We therefore believe that we have evidence indicating that Pizotifen and related compounds are potential disease modifying drugs for the treatment of HD.

Accordingly, in certain embodiments, methods are provided for ameliorating one or more symptoms of Huntington's disease in a mammal. The methods typically involve administering to the mammal one or more agents selected from the group consisting of loxapine, a lopaxine analogue, cyproheptadine, and a cyproheptadine analogue in an amount sufficient to mitigate one ore more symptoms of the disease.

In addition, it is believed these agents are also effective in a prophylactic context. Accordingly, in certain embodiments, prophylactic methods are provided that reduce the risk, and/or lessen the severity, and/or delay the onset or progression of Huntington's disease in a mammal. The methods typically involve administering to the mammal an effective regime of one or more agents selected from the group consisting of loxapine, a lopaxine analogue, cyproheptadine, and a cyproheptadine analogue thereby reducing the risk, lessening the severity, and/or delaying the progression or onset of the disease.

Huntington's disease produces three types of symptoms: movement, cognitive, and psychiatric. The sequence in which symptoms develop varies from person to person. Huntington's related movement disorders, include, but are not limited to uncontrolled movement or tics for example in the fingers, feet, face, or trunk. This is the beginning stage of chorea—involuntary, rapid, ceaseless movement. Chorea can become more intense when the person is anxious or disturbed. Over time other symptoms, such clumsiness, jaw clenching (bruxism), loss of coordination and balance, slurred speech, swallowing and/or eating difficulty, uncontrolled continual muscular contractions (dystonia), walking difficulty, stumbling, falling may emerge.

Over time judgment, memory, and other cognitive functions begin to deteriorate into dementia. As Huntington's disease progresses, the ability to concentrate becomes more difficult. The person may have difficulty driving, keeping track of things, making decisions, answering questions, and may lose the ability to recognize familiar objects.

Early psychiatric symptoms of Huntington's disease are subtle, varied, and easily overlooked or misinterpreted. Depression is the most common psychiatric symptom of Huntington's and often develops early in the course of the disease. Signs of depression include: hostility/irritability, inability to take pleasure in life (anhedonia), and lack of energy. Some people develop manic-depression, or bipolar disorder, during the course of the disease. A person with Huntington's also may exhibit psychotic behavior including, but not limited to delusions, hallucinations, inappropriate behavior (e.g., unprovoked aggression), and paranoia.

In late-onset disease (after age 50), the patient may suffer depression rather than experience sudden anger or irritability, and their memory, reasoning, and problem-solving skills may remain sharp

Early signs of juvenile Huntington's disease often include subtle changes in handwriting and/or a rapid decline in school performance. The child may develop seemingly minor movement disorders, such as slowness, rigidity, tremor, or rapid muscle twitching. Other early signs of disease may include these changes: behavioral changes, difficulty learning new things, and/or speech difficulties. Children under the age of about 15 may experience recurrent seizures and akinesia—muscle rigidity and stiffness. Children from about 15 to about 18 years of age tend to manifest the same symptoms of Huntington's disease as adults.

In addition, lack of physical activity, dietary problems, and eating and swallowing problems can cause constipation, incontinence, and weight loss. Psychiatric and cognitive problems can lead to social isolation and deep depression.

In a therapeutic context, administration of one or more of the active agents described herein (e.g., serotonin antagonists such as loxapine and/or loxapine analogues, and/or cyproheptadine and/or cyproheptadine analogues) can ameliorate any one or more of the symptoms described above. In a prophylactic context, administration of one or more of the active agents described herein (e.g., serotonin antagonists such as loxapine and/or loxapine analogues, and/or cyproheptadine and/or cyproheptadine analogues) can reduce the risk, and/or lessen the severity, and/or delay the onset or progression of the disease.

### Therapeutic Methods.

In various embodiments one or more of the active agents described herein (e.g., serotonin receptor antagonists) are administered to a subject to ameliorate one or more symptoms of Huntington's disease and/or to inhibit progression or severity of such symptoms. In certain embodiments administration of one or more of the active agents described herein (e.g., serotonin antagonists such as loxapine and/or loxapine analogues, and/or cyproheptadine and/or cyproheptadine analogues) can ameliorate any one or more of the symptoms described above. Typically, in a therapeutic context, the active agent(s) described herein are administered to a subject (e.g., an adult human male or female, an adolescent human male or female, or a male or female human child) diagnosed with Huntington's disease.

In certain embodiments amelioration of one or more of the above-identified symptoms is excluded. In certain embodiments treatment of choreoathetosis of Huntington's disease and/or a Huntington's related sleep disorder is excluded.

In various embodiments the subject (e.g., child, adolescent, or adult human male or female) may already exhibit symptoms of Huntington's disease or be diagnosed as having Huntington's disease (e.g., showing >40 CAG repeats in a genetic screen). In such cases, administration of the serotonin receptor antagonists (e.g., loxapine and/or loxapine analogues, and/or cyproheptadine and/or cyproheptadine analogues) can reverse or delay progression of and/or reduce the severity of disease symptoms.

In certain embodiments the effectiveness of treatment can be determined by comparing a baseline measure of a parameter of the disease before administration of serotonin receptor antagonist (e.g., loxapine and/or loxapine analogue, and/or cyproheptadine and/or cyproheptadine analogue) is commenced to the same parameter a one or more timepoints after administration of the active agent(s). Illustrative parameters that can be measured include one or more of the motor, cognitive, or psychiatric symptoms described above, and/or degree of cell death. Mitigation of one or more of the symptoms and/or a decline or reversal of neural cell death indicates partial, substantial, or full efficacy.

In certain embodiments effectiveness of treatment can be evaluated by comparison to the normative time course of the pathology in typical similarly situated subjects (e.g., similar age, severity at first presentation, and the like).

### Prophylactic Methods.

In a prophylactic context, it is contemplated that administration of one or more of the active agents described herein (e.g., serotonin antagonists such as loxapine and/or loxapine analogues, and/or cyproheptadine and/or cyproheptadine analogues) can reduce the risk, and/or lessen the severity, and/or delay the onset or progression of the disease. Typically, in a prophylactic context the active agent(s) described herein are administered to a subject (e.g., an adult human male or female, an adolescent human male or female, or a male or female human child) diagnosed/identified as “at risk” for Huntington's disease. The subject may be asymptomatic, but have one or more environmental, life-style or genetic risk factors, as described herein, and/or be of a defined threshold age. In such instances risk can be identified by any of a number of methods well known to those of skill in the art (e.g., clinicians). Thus, for example, risk can be determined by genetic screening (e.g., screening for mutations in the Huntingtin gene) or by familial history (e.g., presence of the disease or familial risk for the disease), of the subject of interest or of relatives. Genetic testing typically involves analyzing the number of CAG repeats on the huntingtin gene (see, e.g., Table 1).

TABLE 1
Typical reporting for risk for Huntington's disease.

| No. of CAG | |
|---|---|
| Repeats | Outcome |
| <26 | Normal range; individual will not develop HD |
| 27-35 | Individual will not develop HD, but the next generation is |
| | at risk |
| 36-39 | Some, but not all, individuals will develop HD; next |
| | generation is also at risk |
| >40 | Individual will develop HD |

Accordingly, in certain embodiments, a subject may be treated prophylactically where their parent has 27-35 CAG repeats, where the subject has 36-39, or 40 or more CAG repeats.

In certain embodiments prophylaxis for one or more of the above-identified symptoms is excluded. In certain embodiments prophylaxis of choreoathetosis of Huntington's disease and/or a Huntington's related sleep disorder is excluded.

In certain embodiments the methods exclude subjects under treatment for, or identified as at risk for one or more conditions including, but not limited to allergy, headache or migraine, depression, Parkinson's disease, schizophrenia, psychosis or other neuropsychiatric disorder, and/or a sleep disorder. In certain embodiments the subject does not have a neurological disease or disorder other than Huntington's disease. In certain embodiments the mammal does not have a Huntington's related sleep disorder. In certain embodiments the subject mammal is not diagnosed as having, or at risk for, a neurological disease or disorder other than Huntington's disease.

Measurable parameters for evaluating the effectiveness of the prevention regime are as discussed herein for therapy and monitoring.

### Active Agents—Serotonin Receptor Antagonists.

In certain embodiments the prophylactic and/or therapeutic methods described herein involve the use of one or more serotonin receptor antagonists (e.g., at the 5-HT1 and/or 5-HT2A receptor). In various embodiments the serotonin receptor antagonists include, but are not limited to loxapine, and/or a loxapine analogue, and/or cyproheptadine, and/or a cyproheptadine analogue (e.g., pizotifen).

In this regard, it is noted that loxapine and a number of loxapine analogues as well as cyproheptadine and cyproheptadine analogs are commercially available (e.g., as FDA approved pharmaceuticals). Thus, for example, loxapine is available as LOXITANE®, approved for treatment of schizophrenia. Cyproheptadine is available as PERIACTIN®, approved for treatment of allergies, and pizotifen is commercially available as PIZOTYLINE® AND SANDOMIGRAN®, approved for the treatment of migraine headaches.

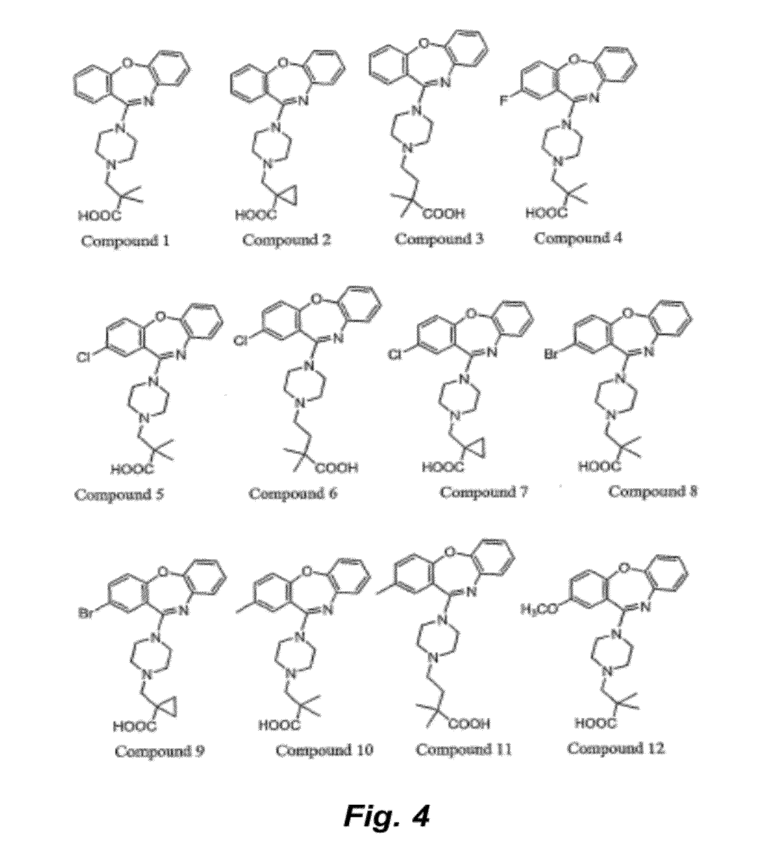

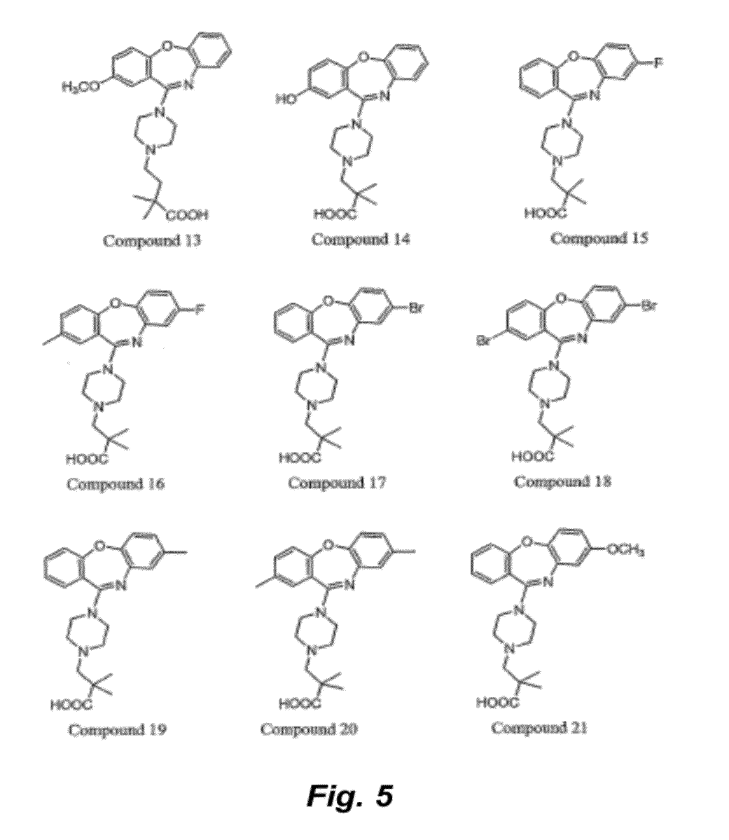

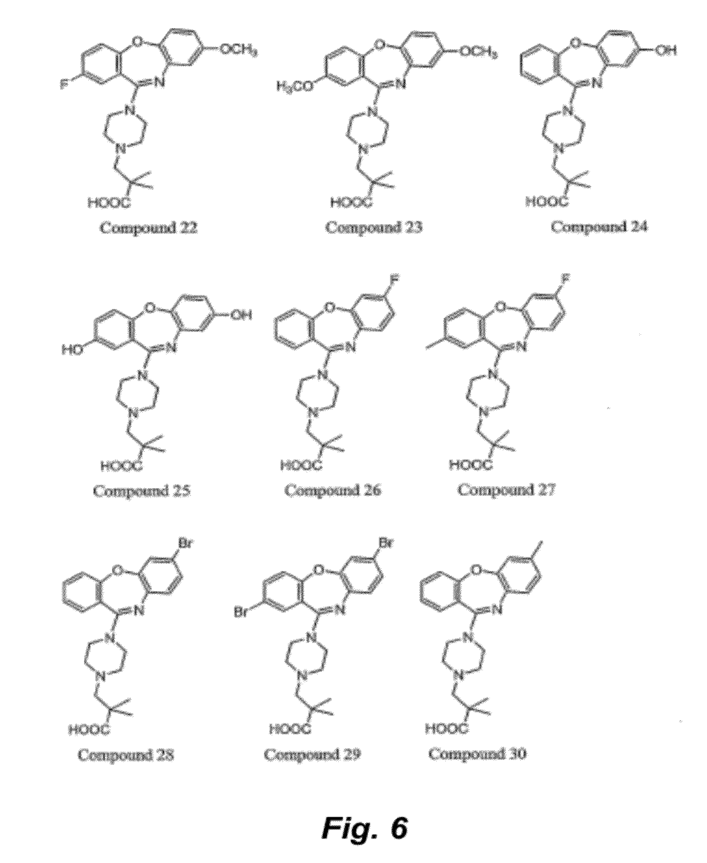

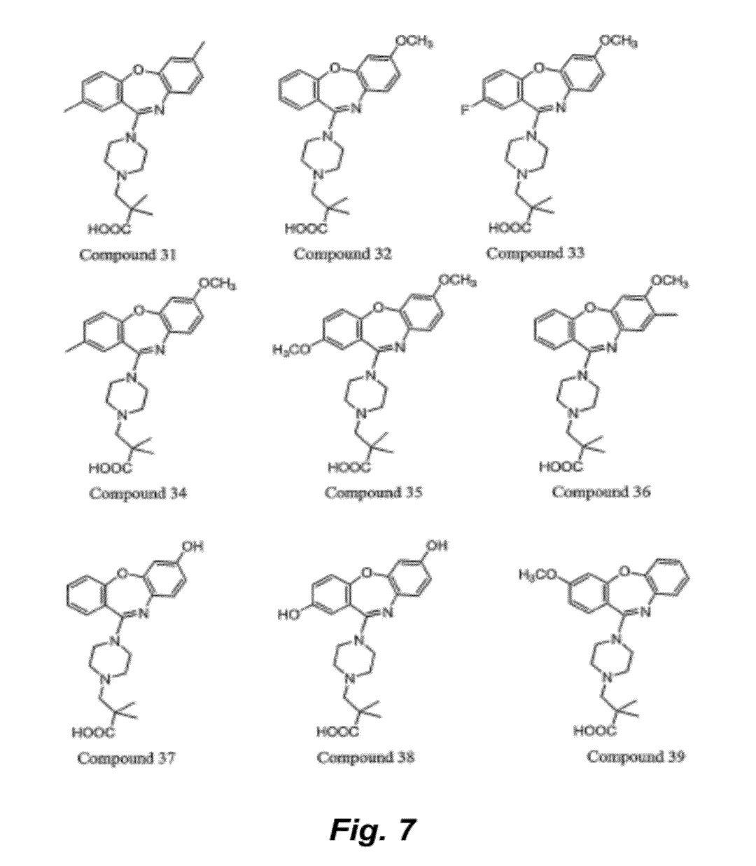

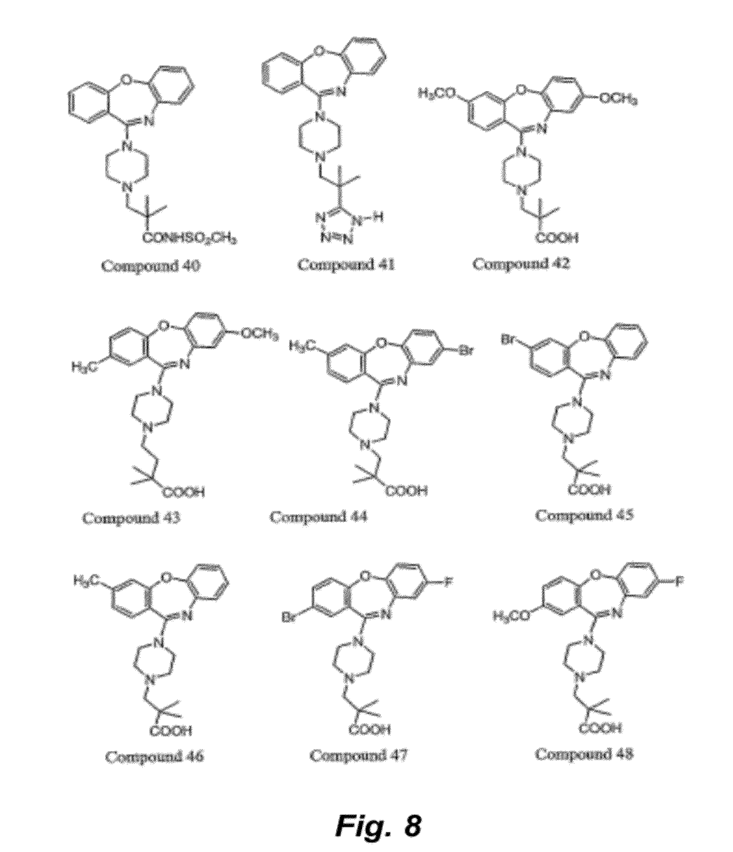

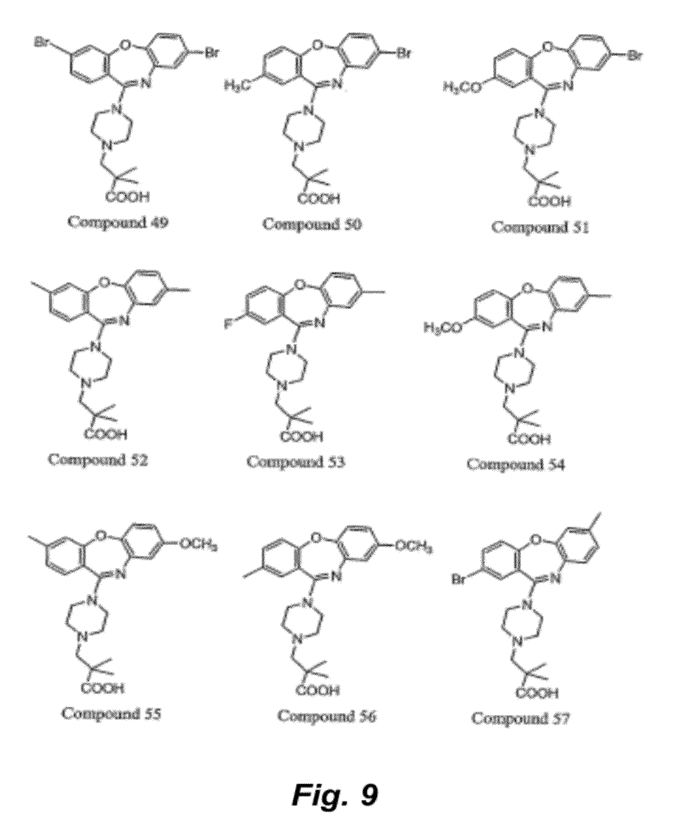

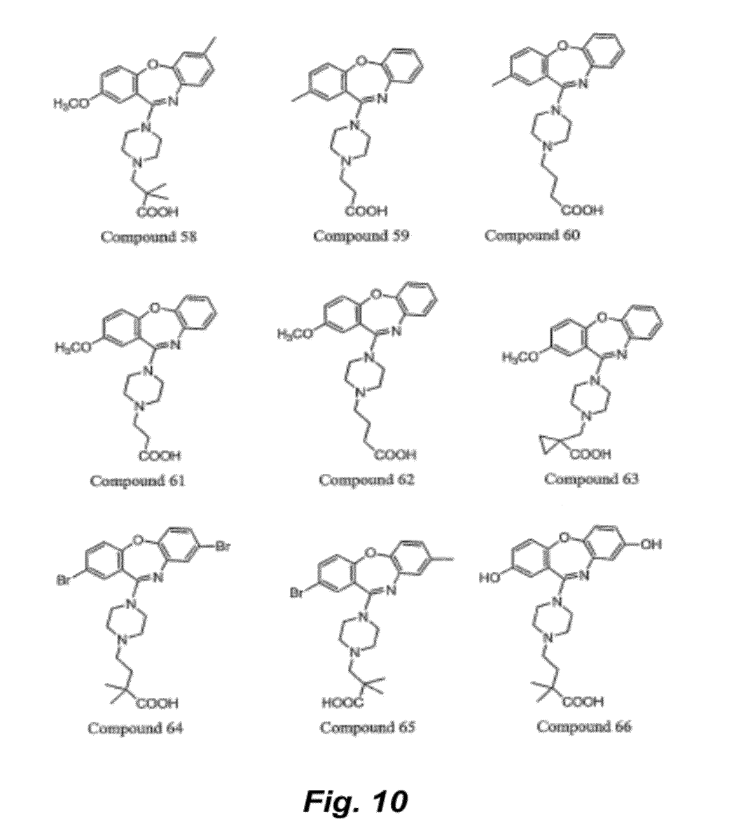

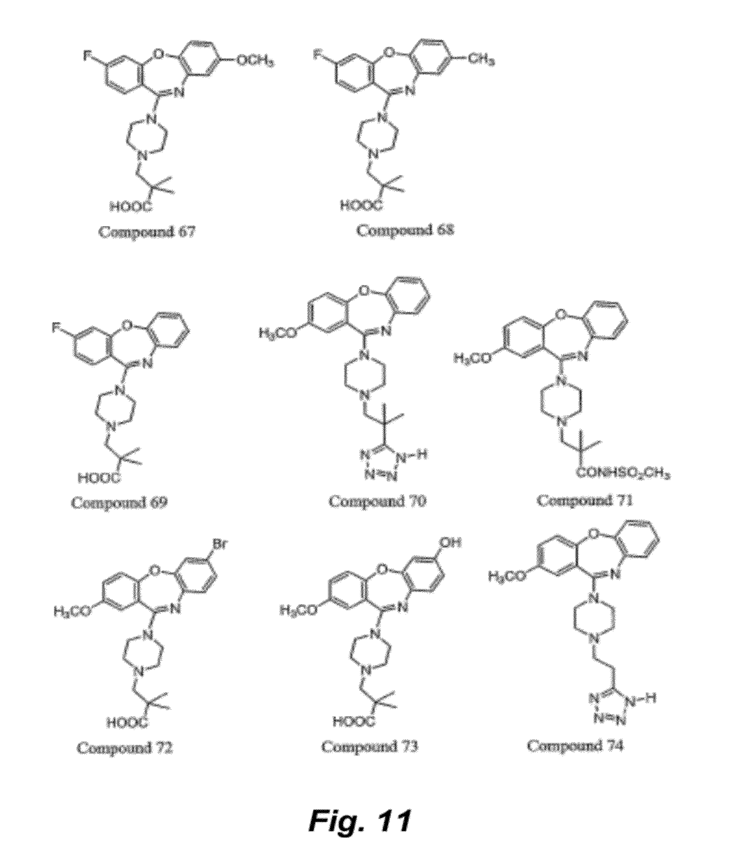

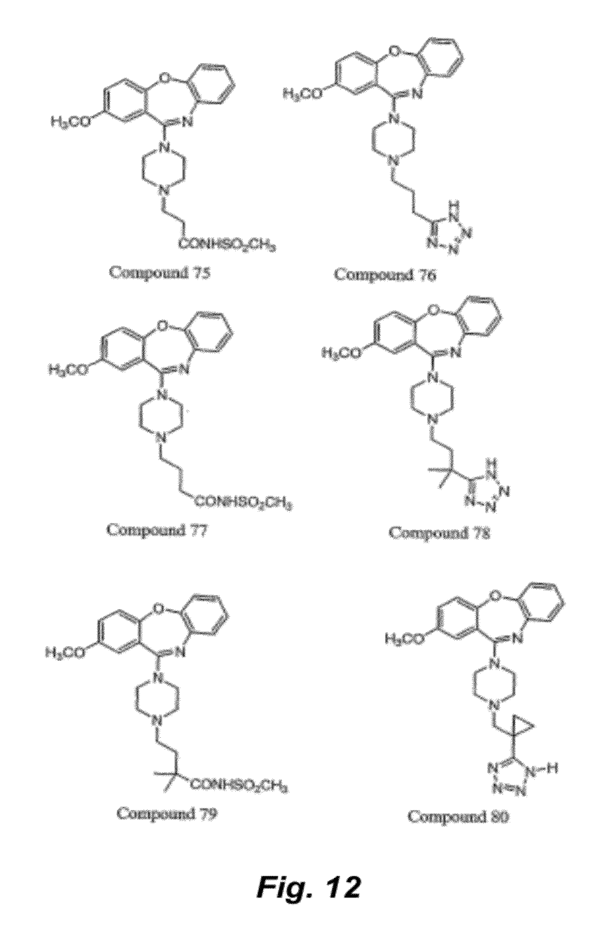

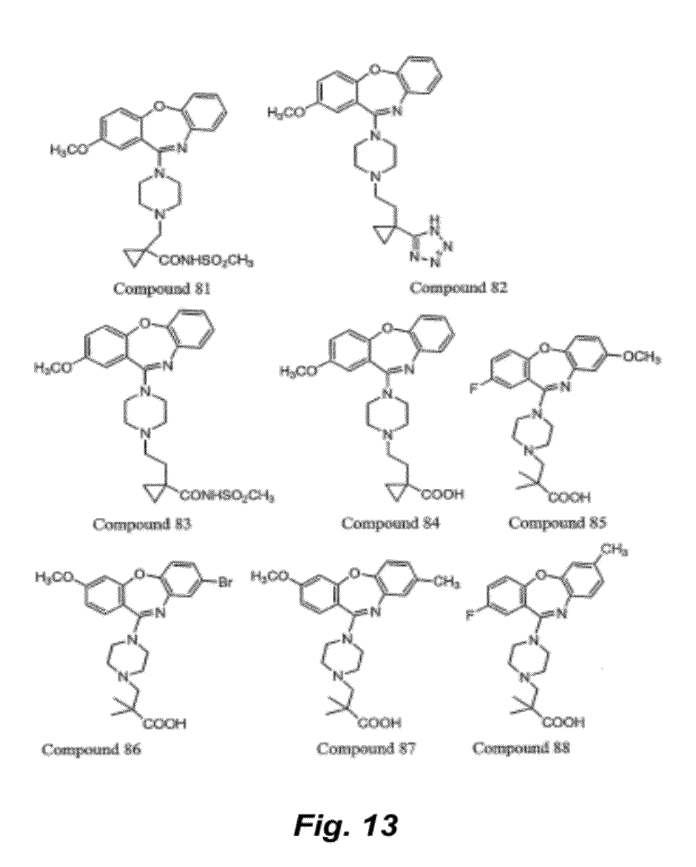

Loxapine analogues and methods of synthesis of such analogues are well known to those of skill in the art. A number of illustrative loxapine analogues and synthesis methods are disclosed in PCT Publication No: WO/2006/034414, which is incorporated herein by reference for the synthesis methods and compounds disclosed therein (see, e.g., Formulas I-IV therein). In various embodiments the analogues include, but are not limited to the compounds shown in FIGS. 4-13 whose synthesis is described in WO/2006/034414, and isoloxapine.

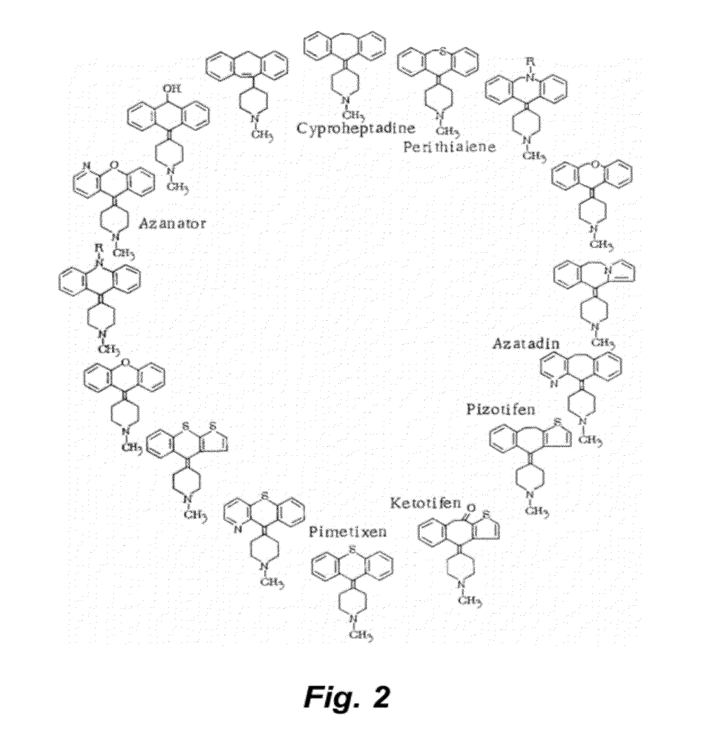

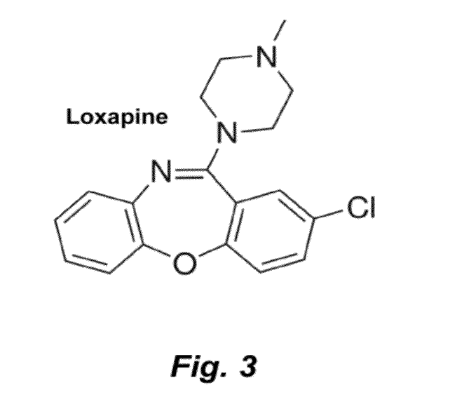

Similarly, cyproheptadine analogues, including pizotifen, and methods of synthesis thereof are well known to those of skill in the art. Illustrative cyproheptadine analogues are shown in FIG. 2, and synthesis of such analogues is described in Vartanyan (1984) Khimiko-farmatsevticheskii Zhurnal (Pharmaceutical Chemistry Journal), 18(11): 736-749.

In various embodiments it is contemplated that to loxapine, cyproheptadine, pizotifen and/or various analogues thereof can be used in combination. Also contemplated are various pharmaceutical forms (e.g., pharmaceutically acceptable salt, ester, solvate, hydrate, or prodrug thereof).

The various active agents are illustrative and not limiting. Using the teachings provided herein the methods can be performed with other analogues as well.

### Administration and Formulations.

In certain embodiments, the active agents described herein (e.g., serotonin receptor antagonists such as loxapine and/or loxapine analogues, and/or cyproheptadine and/or cyproheptadine analogues) are administered to a mammal in need thereof, to a cell, or to a tissue. In various embodiments the compositions can be administered for the treatment or prophylaxis of Huntington's disease.

These active agents can be administered in the “native” form or, if desired, in the form of salts, esters, amides, prodrugs, solvates, hydrates, derivatives, and the like, provided the salt, ester, amide, prodrug, solvate, hydrate, or derivative is suitable pharmacologically, i.e., effective in the present method(s). Salts, esters, amides, prodrugs, solvates, hydrates, and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

Methods of formulating such derivatives are known to those of skill in the art (see, e.g., Remington's Pharmaceutical Science, 15th ed., Mack Publishing Company, Easton, Pa. (1980), Remington: The Science and Practice of Pharmacy, 21st Ed. 2005, Lippincott Williams & Wilkins, and the like).

By way of illustration, acid salts of the therapeutic or prophylactic agents can be prepared from the free base using conventional methodologies that typically involve reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain preferred acid addition salts of the active agents herein include, but are not limited to, halide salts, such as may be prepared using hydrochloric or hydrobromic acids.

Preparation of basic salts of the active agents described herein are prepared in a similar manner using a pharmaceutically acceptable base such as sodium hydroxide, potassium hydroxide, ammonium hydroxide, calcium hydroxide, trimethylamine, or the like. In certain embodiments basic salts include alkali metal salts, e.g., the sodium salt, and copper salts.

For the preparation of salt forms of basic drugs, the pKa of the counterion is preferably at least about 2 pH lower than the pKa of the drug. Similarly, for the preparation of salt forms of acidic drugs, the pKa of the counterion is preferably at least about 2 pH higher than the pKa of the drug. This permits the counterion to bring the solution's pH to a level lower than the pHmax to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (i.e., break down into the individual entities of drug and counterion) in an aqueous environment.

Preferably, the counterion is a pharmaceutically acceptable counterion. Suitable anionic salt forms include, but are not limited to acetate, benzoate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

In various embodiments preparation of esters typically involves functionalization of hydroxyl and/or carboxyl groups that are present within the molecular structure of the active agent. In certain embodiments, the esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

In various embodiments, the active agents identified herein are useful for parenteral, topical, oral, nasal (or otherwise inhaled), rectal, or local administration, such as by aerosol or transdermally, for the prophylaxis and/or treatment of Huntington's disease. The compositions can be administered in a variety of unit dosage forms depending upon the method of administration. Suitable unit dosage forms, include, but are not limited to powders, tablets, pills, capsules, lozenges, suppositories, patches, nasal sprays, injectibles, implantable sustained-release formulations, lipid complexes, etc.

In various embodiments the active agents described herein (e.g., serotonin receptor antagonists such as loxapine and/or loxapine analogues, and/or cyproheptadine and/or cyproheptadine analogues) can be combined with a pharmaceutically acceptable carrier (excipient) to form a pharmacological composition. In certain embodiments, pharmaceutically acceptable carriers include those approved by a regulatory agency of the Federal or a state government or listed in the U.S. Pharmacopeia or other generally recognized pharmacopeia for use in/on animals, and more particularly in/on humans. A “carrier” refers to, for example, a diluent, adjuvant, excipient, auxiliary agent or vehicle with which an active agent of the present invention is administered.

Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds, particularly of use in the preparation of tablets, capsules, gel caps, and the like include, but are not limited to binders, diluent/fillers, disentegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g. calcium carbonate, carboxymethylcellulose calcium, sodium starch glycolate, crospovidone etc.), a binder (e.g. alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the active component or components (e.g., active peptide) and the resulting composition is compressed. Where necessary the compressed product is coated, e.g., known methods for masking the taste or for enteric dissolution or sustained release. Suitable coating materials include, but are not limited to ethyl-cellulose, hydroxymethylcellulose, polyoxyethylene glycol, cellulose acetate phthalate, hydroxypropylmethylcellulose phthalate, and Eudragit (Rohm & Haas, Germany; methacrylic-acrylic copolymer).

In certain embodiments the active agent(s) described herein can be delivered in a nano crystal dispersion formulation. Preparation of such formulations is described, for example, in U.S. Pat. No. 5,145,684. Illustrative nanocrystalline dispersions of HIV protease inhibitors and their method of use are described in U.S. Pat. No. 6,045,829. The nanocrystalline formulations typically can often afford greater bioavailability of drug compounds.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain therapeutic applications, the compositions of this invention are administered therapeutically to a patient suffering from Huntington's disease or prophylactically to a subject at risk for HD in an amount sufficient to prevent and/or cure and/or at least partially prevent or arrest the disease and/or its complications. An amount adequate to accomplish this is defined as a “therapeutically effective dose.” An amount effective to inhibit the onset and/or progression and/or severity of the disease is a prophylactically effective amount. Amounts effective for therapeutic or prophylactic use will depend upon the severity of the disease and the general state of the patient's health. Single or multiple administrations of the compositions may be utilized depending on the dosage and frequency as required and tolerated by the patient. In any event, the composition should provide a sufficient quantity of the active agents of the formulations of this invention to effectively treat (ameliorate one or more symptoms in) the patient or to afford partial or full prophylaxis.

The concentration/amount of active agent(s) in a formulation can vary widely, and will be selected primarily based on activity of the active ingredient(s), body weight and the like in accordance with the particular mode of administration selected and the patient's needs. Concentrations, however, will typically be selected to provide dosages ranging from about 0.1 or 1 mg/kg/day to about 50 mg/kg/day and sometimes higher. Typical dosages range from about 3 mg/kg/day to about 3.5 mg/kg/day, preferably from about 3.5 mg/kg/day to about 7.2 mg/kg/day, more preferably from about 7.2 mg/kg/day to about 11.0 mg/kg/day, and most preferably from about 11.0 mg/kg/day to about 15.0 mg/kg/day. In certain preferred embodiments, dosages range from about 10 mg/kg/day to about 50 mg/kg/day. In certain embodiments, dosages range from about 20 mg to about 50 mg given orally twice daily. It will be appreciated that such dosages may be varied to optimize a therapeutic and/or prophylactic regimen in a particular subject or group of subjects.

In various embodiments, the active agents described herein can be administered parenterally, for example, by IV, IM, depo-IM, SC, or depo-SC. When administered parenterally, a therapeutically effective amount of about 0.5 to about 100 mg/day, preferably from about 5 to about 50 mg daily should be delivered. When a depot formulation is used for injection once a month or once every two weeks, the dose should be about 0.5 mg/day to about 50 mg/day, or a monthly dose of from about 15 mg to about 1,500 mg. In part because of the forgetfulness of the patients with Huntington's disease, in certain embodiments, it is preferred that the parenteral dosage form be a depo formulation.

An illustrative, but non-limiting, typical adult dose for pizotifen is 1.5 mg at night or 500 micrograms 3 times daily, adjusted according to response. A typical maximum single dose is 3 mg, and a typical maximum daily dose is 4.5 mg.

In various embodiments, the active agents described herein can be administered sublingually. In certain embodiments when given sublingually, the active agents described herein analog can be given one to six times daily in the amounts described above.

In various embodiments, the active agents described herein can be administered intranasally. When given by this route, the typical dosage forms include, but are not limited to a nasal spray or dry powder, as is known to those skilled in the art. In certain embodiments the dosage of the active agents described herein for intranasal administration is the amount described above for.

In various embodiments, the active agents described herein can be administered intrathecally. When given by this route the appropriate dosage form can be a parenteral dosage form as is known to those skilled in the art. In certain embodiments the dosage of the active agents described herein for intrathecal administration is the amount described above for IM administration.

In certain embodiments, the active agents described herein can be administered topically. When given by this route, the suitable dosage forms include, but are not limited to a cream, ointment, or patch. Because the amount that can be delivered by a patch is limited, two or more patches may be used. The number and size of the patch is not important, what is important is that a therapeutically effective amount of active agent be delivered as is known to those skilled in the art. The active agents described herein can be administered rectally by suppository as is known to those skilled in the art.

In various embodiments, the active agents described herein can be administered by implants or metering systems/pumps as is known to those skilled in the art.

It should be apparent to one skilled in the art that the exact dosage and frequency of administration will depend on the particular condition being treated, the severity of the condition being treated, the age, weight, general physical condition of the particular patient, and other medication the individual may be taking as is well known to administering physicians who are skilled in this art.

Combination Therapies

In various embodiments the active agent(s) described herein can be used in combination with other therapeutic agents or approaches used to treat or prevent Huntington's disease. Typically, such medications include, but are not limited to various medications to help control emotional and movement problems. Such therapeutic agents include, but are not limited to antipsychotics for treatment of hallucinations, delusions, and violent outbursts. Suitable antipsychotics include but are not limited to haloperidol, chlorpromazine, olanzapine (contraindicated if patient has dystonia). In various embodiments antidepressants are administered for depression, and/or obsessive-compulsive behavior). Illustrative antidepressants include, but are not limited to fluoxetine, sertraline hydrochloride, and nortriptyline. In various embodiments tranquilizers are used for the treatment of anxiety and chorea. Illustrative tranquilizers include, but are not limited to benzodiazepines, paroxetine, venlafaxin, and beta-blockers. Mood stabilizers (e.g., lithium, valproate, carbamazepine, and the like) can be used for the treatment of mania and/or bipolar disorder. In certain embodiments Botulinum toxin is administered for dystonia, jaw clenching. Additionally, tetrabenazine (Xenazine) is approved by the U.S. Food and Drug Administration (FDA) to treat chorea associated with Huntington's disease. This drug usually is administered once per day to start, and the dosage may be gradually increased to up to three times a day. Side effects include excessive sleepiness (somnolence), fatigue, nausea, and restlessness. Tetrabenazine also may increase the risk for depression and suicidal thoughts or behavior (suicidality).

Because most drugs used to treat the symptoms of HD can produce undesirable side effects, ranging from fatigue to restlessness and hyperexcitability, physicians often prescribe the lowest possible dose.

### Other CAG Repeat Diseases.

While the therapeutic and prophylactic methods described herein are discussed with reference to Huntington's disease, in certain embodiments, it is contemplated that the same therapeutic and/or prophylactic methods and compounds (e.g., serotonin receptor antagonists such as loxapine, and/or a loxapine analogue, and/or cyproheptadine, and/or a cyproheptadine analogue (e.g., pizotifen)).

In a coding region the repeated CAG codon codes for glutamine (Q), resulting in a polyglutamine tract. These CAG-repeat diseases are also commonly referred to as polyglutamine (or PolyQ) diseases. A number of illustrative polyQ diseases are listed in Table 2. In various embodiments it is contemplated that the therapeutic and/or prophylactic methods described herein are effective for any one or more of the diseases listed in Table 2

TABLE 2
Illustrative polyglutamine (PolyQ) diseases.

| | | | Patho- |
|---|---|---|---|
| | | Normal | genic |
| | | PolyQ | PolyQ |
| | Gene | repeats | repeats |
| DRPLA | ATN1 or | 6-35 | 49-88 |
| (Dentatorubropallidoluysian | DRPLA | | |
| atrophy) | | | |
| HD | HTT | 10-35 | 35+ |
| (Huntington's disease) | (Huntingtin) | | |
| SBMA | Androgen | 9-36 | 38-62 |
| (Spinobulbar muscular atrophy or | receptor on the X | | |
| Kennedy disease) | chromosome. | | |
| SCA1 | ATXN1 | 6-35 | 49-88 |
| (Spinocerebellar ataxia Type 1) | | | |
| SCA2 | ATXN2 | 14-32 | 33-77 |
| (Spinocerebellar ataxia Type 2) | | | |
| SCA3 | ATXN3 | 12-40 | 55-86 |
| (Spinocerebellar ataxia Type 3 or | | | |
| Machado-Joseph disease) | | | |
| SCA6 | CACNA1A | 4-18 | 21-30 |
| (Spinocerebellar ataxia Type 6) | | | |
| SCAT | ATXN7 | 7-17 | 38-120 |
| (Spinocerebellar ataxia Type 7) | | | |
| SCA17 | TBP | 25-42 | 47-63 |
| (Spinocerebellar ataxia Type 17) | | | |

### Kits.

In certain embodiments kits are provided for the prophylaxis and/or treatment of Huntington's disease. The kits typically comprise a container containing one or more of the active agents described herein (e.g., serotonin receptor antagonists such as loxapine and/or loxapine analogues, and/or cyproheptadine and/or cyproheptadine analogues). In certain embodiments the active agent(s) can be provided in a unit dosage formulation (e.g., suppository, tablet, caplet, patch, etc.) and/or may be optionally combined with one or more pharmaceutically acceptable excipients.

In addition, the kits optionally include labeling and/or instructional materials providing directions (i.e., protocols) for the practice of the prophylactic or therapeutic methods described herein. Certain instructional materials describe the use of one or more active agent(s) described herein therapeutically or in the prophylaxis of Huntington's disease. The instructional materials may also, optionally, teach preferred dosages/therapeutic regiment, counter indications and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### The Serotonin Receptor Antagonists Loxapine, Cyproheptadine and Pizotifen are Potential Disease-Modifying Drugs for the Treatment or Prophylaxis of Huntington's Disease

In order to identify approved drugs that could have disease-modifying activity in Huntington's disease (HD), we used a mammalian cell-based assay of mutant huntingtin-mediated cell death to screen the Prestwick Chemical library. This library is composed of 1,200 marketed drugs with favorable safety, bioavailability and efficacy properties. Our screen identified several compounds that could reproducibly suppress huntingtin-mediated cell death in a dose-dependent manner. Strikingly, three of these hits Loxapine, Cyproheptadine and Pizotifen are approved serotonin receptor antagonist drugs. In an independent genome-scale RNAi screen for suppressors of cell death using this same cell-based model, we also obtained the targets of these hits as modifiers including HTR2C and HTR1F. These data demonstrate that serotonin receptors and ligands are relevant to HD. On the basis of these observations, we selected Pizotifien for testing in the R6/2 mouse model of HD. Pizotifen was selected because of its safety profile and CNS penetrance. In two independent studies, we observed that this drug was able to robustly correct motor deficits (i.e. Rotarod performance) in R6/2 to near wild-type levels. Open field behavior analysis indicated that the general activity level of treated animals was normal. We therefore believe that we have evidence indicating that Pizotifen and related compounds are potential disease modifying drugs for the treatment of HD.

### Results

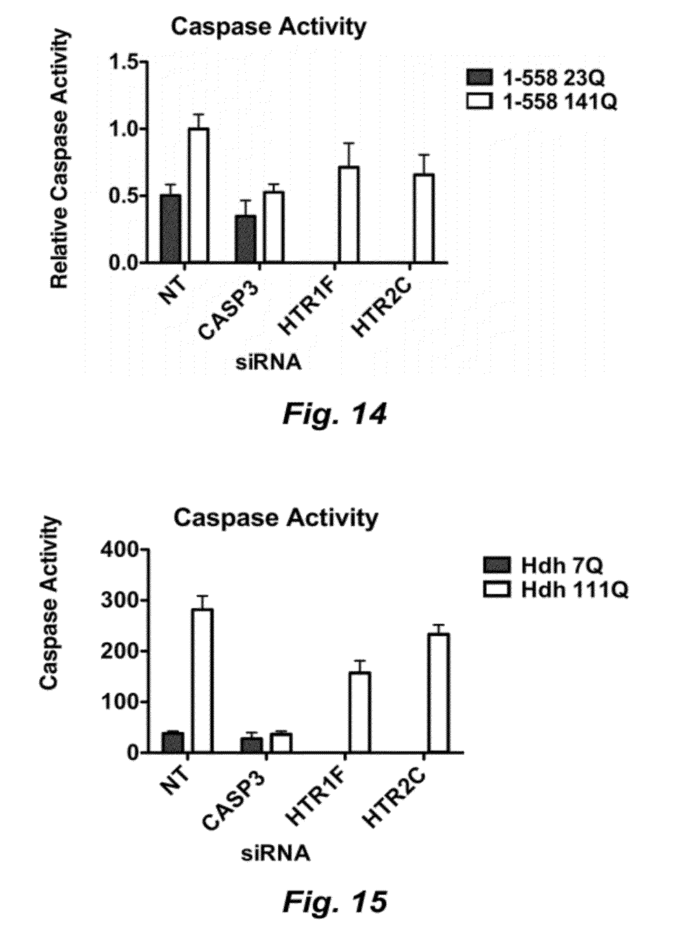

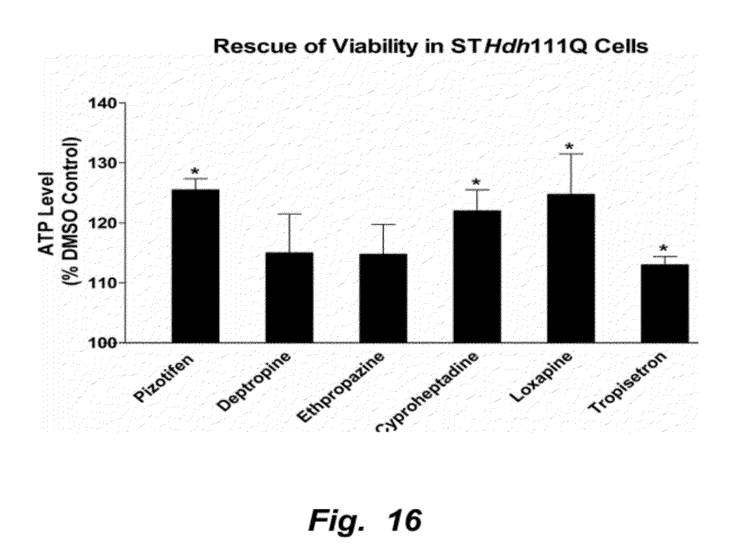

As part of a comprehensive HD target discovery and validation program, we have completed a screen of >7,200 siRNA pools corresponding to a druggable genome (Dharmacon). This screen was designed to detect loss-of-function suppressors of cell death in HEK293T cells expressing an expanded (141Q) mutant huntingtin (Htt 1-558) N-terminal fragment. Two related targets identified in druggable genome RNAi screen are the serotonin receptors (5-HTRs) e.g. HTR2C and HTR1F (FIG. 14). This result was confirmed in the STHdh^{111Q/111Q} cells (FIG. 15).

Suppressions by RNAi mediated loss-of-function in 5-HTRs is validated by observing effects of 5-HTR antagonists in cell based assays. In a parallel unbiased chemical modifier screen we used the same STHdh111Q cell-based assay to screen a small (1120 compound) library of approved drugs and pharmacologically active compounds (Prestwick Library) for small molecules that could suppress caspase activation and cell death mediated by serum deprivation. Among the confirmed hits from this screen were six 5-HTR antagonists. Activities of the top six hits from the Prestwick Library screen in an ATP assay are shown in FIG. 16. This independent screening result greatly enhanced our confidence that the 5-HTR RNAi results were valid and resulted from on-target RNAi effects on 5HT receptors.

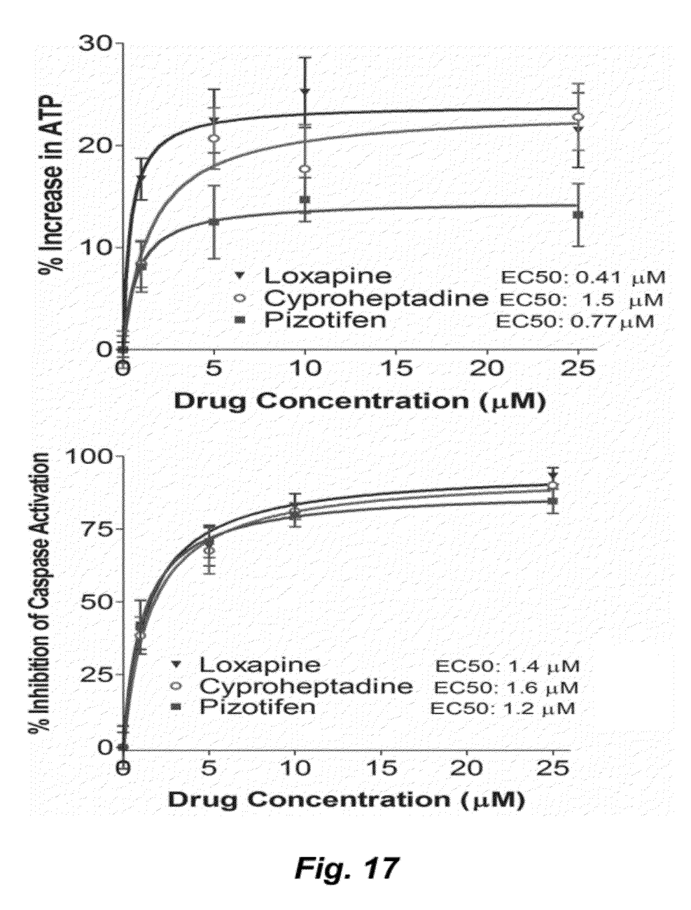

Because of their structural similarity, safety profiles and CNS indications, we performed dose/response studies of the 5-HTR antagonist drugs Loxapine, Cyproheptadine and pizotifen in viability (ATP) and caspase activation assays using the STHdh111Q/111Q serum deprivation model. These results, shown in FIG. 17, demonstrate that these drugs all rescue loss of viability (upper panel) and caspase-3/7 activation (lower panel) in a dose dependent manner.

### Expression of Serotonin Receptors (5-HTR) Targets in HD Models (STHdh111Q and R6/2).

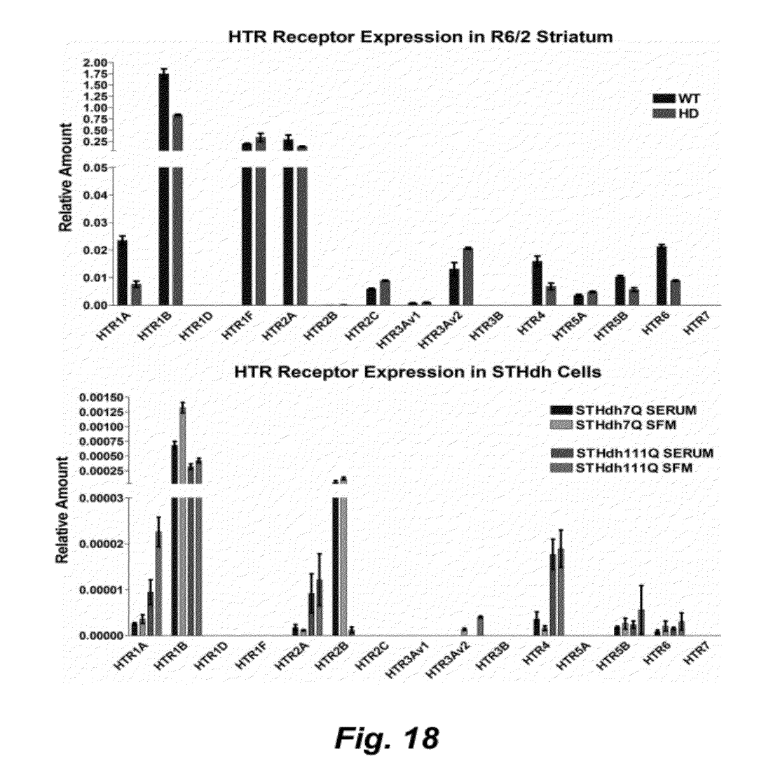

Messenger RNA expression levels of 5HTR family members in STHdh111Q cells and R6/2 striatum were measured using real-time quantitative PCR. In the case of the STHdh111Q cells, we compared expression under normal (serum) and serum deprived (SFM) conditions since these are the conditions we used in out cell-based assays (FIGS. 14-17). In the case of R6/2 we compared transgenic mice to non-transgenic littermate controls to assess the impact of mutant Htt on 5-HTR expression patterns. FIG. 18 shows the results of these studies. It is clear that known targets of Pizotifen, Cyproheptadine and Loxapine are expressed in these models (see Table 3). It is notable that two targets observed as suppressors in the STHdh111Q cells, HTR2C and HTR1F were not detected in this cell type. This may reflect a technical failure or the fact that the RNAi suppressors effects are due to off target effects on other family members.

TABLE 3
Features of 5-HTR antagonists shown in FIG. 16.

| Drug | Indication | Known Targets | Approved |
|---|---|---|---|
| Pizotifen | migraine | 5-HTR1A, 5- | UK, Canada, |
| (Sandomigrin) | | HTR2A, | Australia, Europe |
| | | 5-HTR2C, 5-HTR3 | |
| Cyproheptadine | allergy | 5-HTR1A, 5- | US FDA |
| (Periactin) | | HTR2A, | |
| | | 5-HT2C | |
| Loxapine | schizophrenia | 5-HTR1B, 5- | US FDA |
| (Loxitane) | | HTR2A, | |
| | | 5-HT2RC, 5-HT3, | |
| | | 5-HTR5, | |
| | | 5-HTR6, 5-HTR7 | |

### Pizotifen, a Serotonin Receptor Antagonist, Improves Rotarod Performance in the R6/2 Mouse Model.

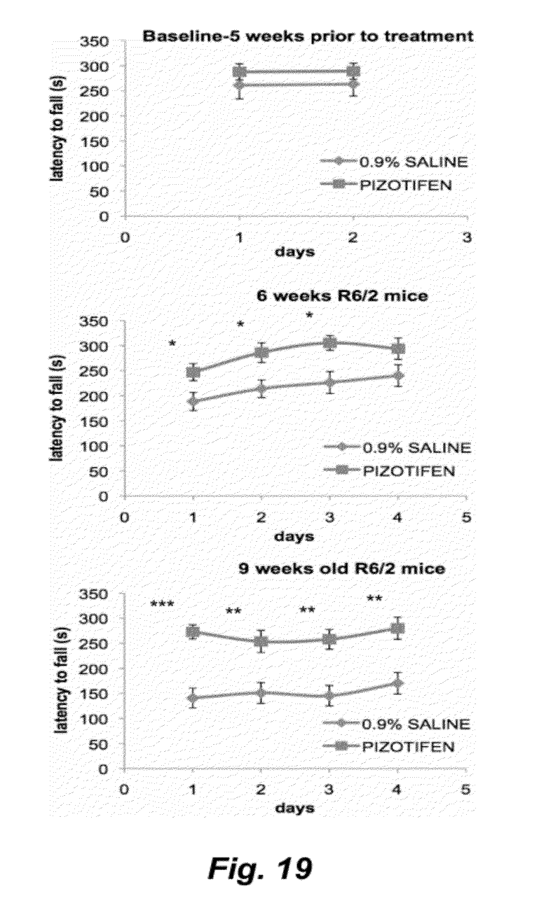

To confirm that 5-HT receptors represent important therapeutic targets in HD, we conducted a therapeutic trial in R6/2 HD mice, treating with pizotifen daily (10 mg/kg IP) starting at 5 weeks of age. In this study, rotarod performance was improved after only one week of treatment and significant beneficial effects were observed at 9 weeks. FIG. 19 shows the results of this preliminary study. As shown in the upper panel, mice in the treatment and control groups start with the same rotarod performance (N=15 saline and N=14 pizotifen group, prior to treatment). Interestingly after three days of treatment the 6-week old group treated with Pizotifin already show a significant improvement in rotorad performance (middle panel). Strikingly, pizotifin corrects the rotarod deficit to near wild-type levels at 9 weeks of age (lower panel). These data confirm our initial observations that 5-HT receptors are targets of interest in HD and that the drug pizotifen (and other 5-HTR antagonists) should be investigated further for potential therapeutic activities in HD). We also measured open field and lifespan in these studies and found no statistical differences in each treatment group. This may reflect a specific mechanism of action for pizotifen.

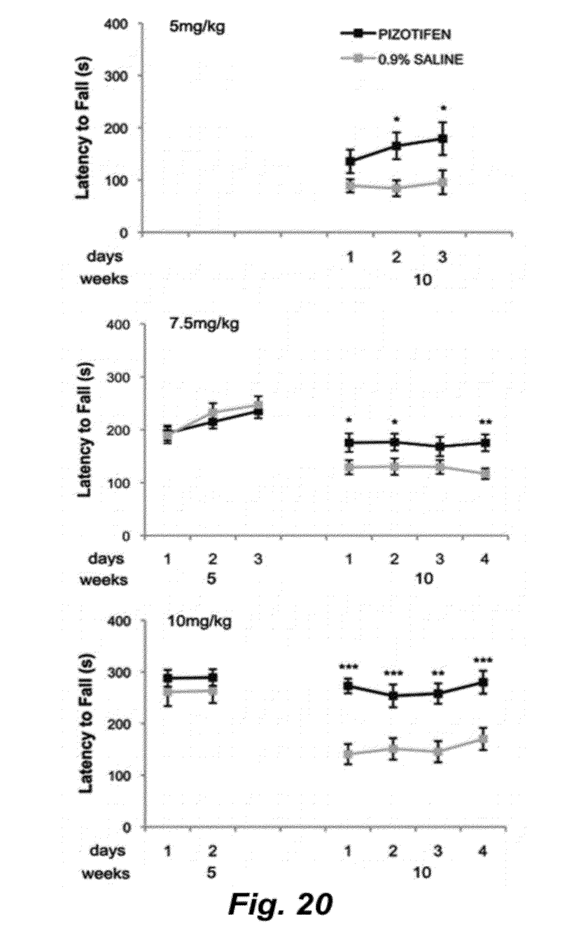

FIG. 20 illustrates a dose-dependent effect of pizotifen on correction of rotarod.

### In Vivo Activity of Pizotifen.

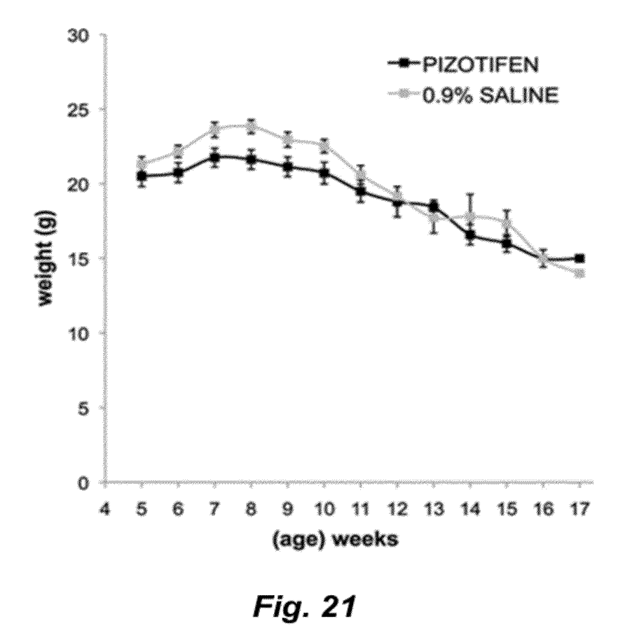

FIG. 21 demonstrates that pizotifen (at 7.5 mg/kg) doesn't improve or worsen weight loss in the mouse model. In particular, it is noted that pizotifen does not appear to be protective against weight loss.

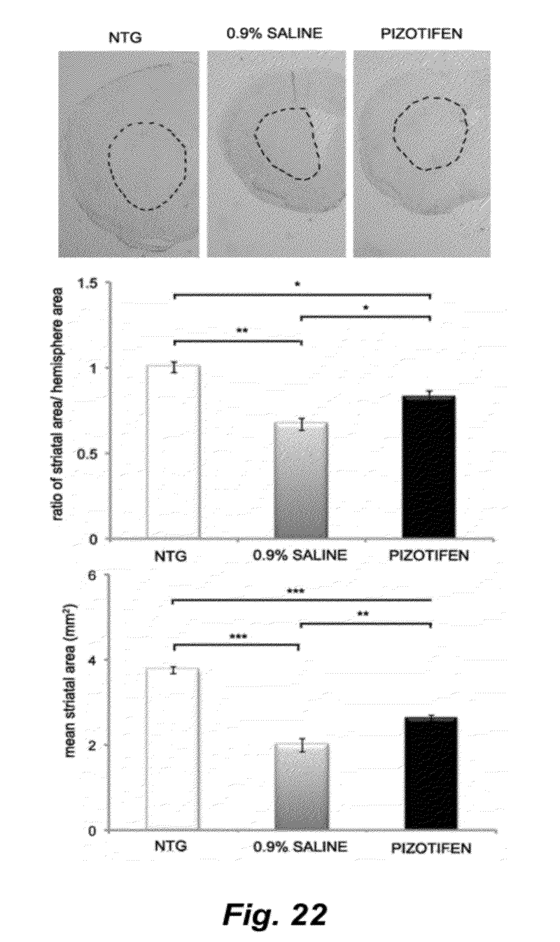

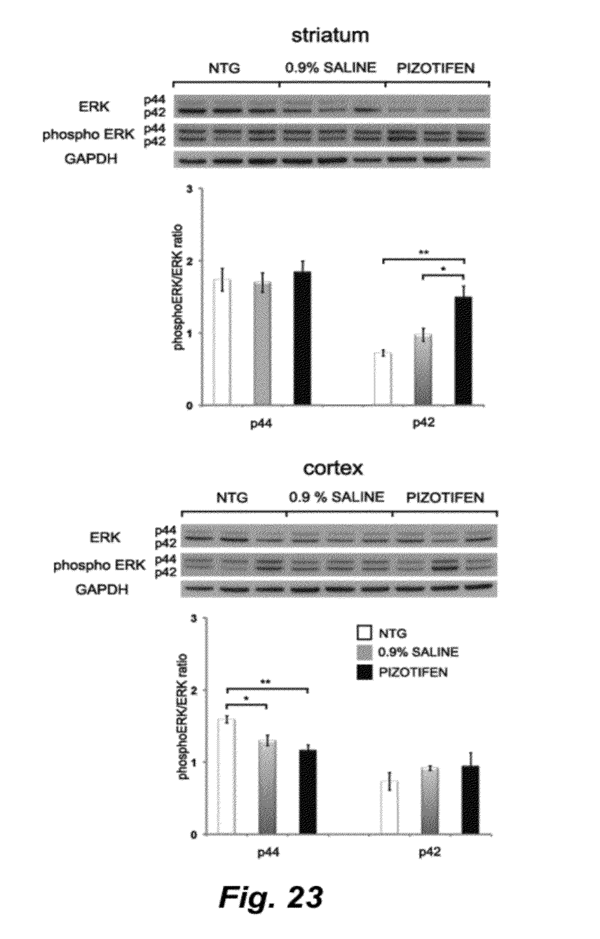

FIG. 22 shows that in vivo pizotifen is effective to preserve striatal area. As illustrated in FIG. 23, one mechanism of action of pizotifen appears to be the activation of the ERK pathway in vivo. This is consistent with our in vitro observations. As illustrated in FIG. 23, this effect was not observed in the cortex and may be striatal dependent.

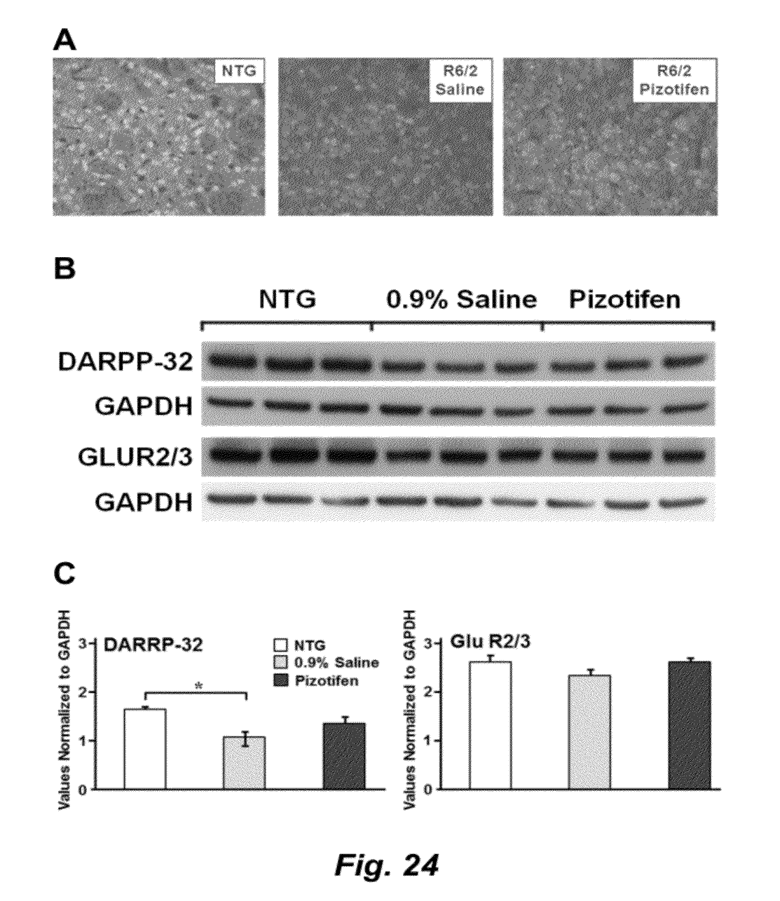

Expression of dopamine- and cAMP-regulated phosphoprotein, 32 kDa (DARPP-32), has been shown to be downregulated in mouse models of HD (see, e.g., Van Dellen et al. (2000) NeuroReport, 11(17): 3751-3757) and DARPP-32 is often used as a measure of disease progression. FIG. 24, panels A-C, show that DARPP-32 is rescued by pizotifen further indicating that pizotifen acts on the underlying disease state.

Activation of the ERK pathway is a plausible mechanism of activity of pizotifen and the other agents described herein. Our evidence shows that in the mouse model motor activity is improved and accompanied by or caused by the preservation (and possible restoration) of neuronal cells in vivo. Our data indicated that the use of the active agent(s) described herein, results in improvement (or at least preservation) of brain physiology/function.—The progression of the disease appears to be slowed or stopped. Accordingly it is believed that the observed improvement in motor function can be attributed to the modification of the underlying disease state. Thus, it is believed that the agents described herein can be particularly useful for the protection or preservation of neuronal function.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of ameliorating one or more symptoms of Huntington's disease in a mammal, said method comprising:
administering to said mammal one or more agents selected from the group consisting of loxapine, a lopaxine analogue, cyproheptadine, and a cyproheptadine analogue in an amount sufficient to mitigate symptoms of the disease.

2. The method of claim 1, wherein said agents comprise cyproheptadine and/or a cyproheptadine analogue.

3. The method of claim 2, wherein said agent comprises cyproheptadine or pizotifen.

4-5. (canceled)

6. The method of claim 2, wherein said agent comprises a compound selected from group consisting of the compounds listed in FIG. 2.

7. (canceled)

8. The method of claim 1, wherein said agents comprise loxapine and/or a loxapine analogue.

9-11. (canceled)

12. The method of claim 8, wherein said loxapine analogue is isoloxapine.

13. The method of claim 8, wherein said loxapine analogue comprises a compound selected from the group consisting of compounds listed in FIGS. 4-13.

14. The method of claim 1, wherein said agent is formulated in a pharmaceutically acceptable excipient.

15. The method of claim 1, wherein said agent is formulated as a pharmaceutically acceptable salt, ester, amide, solvate, hydrate, or prodrug thereof.

16. The method of claim 1, wherein said agent is formulated for delivery via a modality selected from the group consisting of isophoretic delivery, transdermal delivery, aerosol administration, administration via inhalation, oral administration, intravenous administration, and rectal administration.

17. The method of claim 1, wherein said agent is administered via a route selected from the group consisting of isophoretic delivery, transdermal delivery, aerosol administration, administration via inhalation, oral administration, intravenous administration, and rectal administration.

18. The method of claim 1, wherein the mammal is human.

19. The method of claim 18, wherein the mammal is a human diagnosed as having Huntington's disease.

20. The method of claim 18, wherein the mammal is a human having the Huntington mutation.

21. The method of claim 1, wherein said mammal does not have, and/or is not under treatment for, and/or is not identified as at risk for one or more conditions selected from the group consisting of an allergy, headache or migraine, depression, Parkinson's disease, schizophrenia, psychosis or other neuropsychiatric disorders, and a sleep disorder.

22. The method of claim 1, wherein said mammal does not have a neurological disease or disorder other than Huntington's disease and/or said mammal does not have a Huntington's related sleep disorder, and/or said mammal is not diagnosed as having or at risk for a neurological disease or disorder other than Huntington's disease.

23-24. (canceled)

25. The method of claim 1, wherein said ameliorating comprises one or more of the following:
inhibiting neural cell death and/or restoring neurological cell function;
inhibiting the onset, progression, or severity of a Huntington's related movement disorder, and/or a Huntington's related cognitive disorder, and/or a Huntington's related psychiatric disorder;
inhibiting the onset, progression, or severity of one or more symptoms selected from the group consisting of clumsiness, jaw clenching (bruxism), loss of coordination and balance, slurred speech, swallowing and/or eating difficulty, uncontrolled continual muscular contractions (dystonia), walking difficulty, stumbling, and falling, loss of memory, loss of concentration, inability to recognize familiar objects, hostility/irritability, inability to take pleasure in life (anhedonia), lack of energy, delusions, hallucinations, inappropriate behavior (e.g., unprovoked aggression), and paranoia;
an improvement in the cognitive abilities of the mammal; and
a perceived improvement in quality of life by a human subject.

26-31. (canceled)

32. The method of claim 1, wherein the administering is over a period of at least three weeks.

33. (canceled)

34. A method of reducing the risk, lessening the severity, or delaying the onset or progression of Huntington's disease in a mammal, said method comprising:
administering to said mammal an effective regime of one or more agents selected from the group consisting of loxapine, a lopaxine analogue, cyproheptadine, and a cyproheptadine analogue thereby reducing the risk, lessening the severity, and/or delaying the progression or onset of the disease.

35. The method of claim 34, wherein said agents comprise cyproheptadine and/or a cyproheptadine analogue.

36. The method of claim 35, wherein said agent comprises cyproheptadine or pizotifen.

37-38. (canceled)

39. The method of claim 34, wherein said agent comprises a compound selected from group consisting of the compounds listed in FIG. 2.

40. (canceled)

41. The method of claim 34, wherein said agents comprise loxapine and/or a loxapine analogue.

42-44. (canceled)

45. The method of claim 41, wherein said loxapine analogue is isoloxapine.

46. The method of claim 41, wherein said loxapine analogue comprises a compound selected from the group consisting of compounds listed in FIGS. 4-13.

47-68. (canceled)

69. A method of ameliorating one or more symptoms of a disease characterized by poly glutamine repeats, said method comprising:
administering to said mammal one or more serotonin receptor antagonists in an amount sufficient to ameliorate symptoms of the disease.

70. The method of claim 69, wherein said disease is selected from the group consisting of DRPLA (Dentatorubropallidoluysian atrophy), HD (Huntington's disease), SBMA (Spinobulbar muscular atrophy or Kennedy disease), SCAT (Spinocerebellar ataxia Type 1), SCA2 (Spinocerebellar ataxia Type 2), SCA3 (Spinocerebellar ataxia Type 3 or Machado-Joseph disease), SCA6 (Spinocerebellar ataxia Type 6), SCAT (Spinocerebellar ataxia Type 7), and SCA17 (Spinocerebellar ataxia Type 17).

71. The method of claim 69, wherein the disease is Huntington's disease and said agents do not include Risperidone.

72. The method of claim 69, wherein said symptoms do not include choreoathetosis of Huntington's disease.

73. The method of claim 69, wherein said agents are selected from the group consisting of loxapine, a lopaxine analogue, cyproheptadine, and a cyproheptadine analogue.

74. The method of claim 69, wherein said mammal does not have, and/or is not under treatment for, and/or is not identified as at risk for one or more conditions selected from the group consisting of an allergy, headache or migraine, depression, Parkinson's disease, schizophrenia, psychosis or other neuropsychiatric disorders, and a sleep disorder.

75-77. (canceled)
